(19) United States
(12) Patent Application Publication
(10) Pub. No.: US 2007/0038059 A1
(43) Pub. Date: Feb. 15, 2007

(54) Implant and instrument morphing

(76) Inventors: Garrett Sheffer, Hoboken, NJ (US); Lance Perry, Warsaw, IN (US)

Correspondence Address: Intellectual property Group;Bose McKinney & Evans LLP, 2700 First Indiana Plaza, 135 North Pennsylvania St, Indianapolis, IN, 46204, US

(21) Appl. No.: 11/438,886

(22) Filed: May 23, 2006

Related U.S. Application Data
(60) Provisional application No. 60/697,093, filed on Jul. 7, 2005.

(51) Int. Cl. A61B 5/05

(52) U.S. Cl. 600/407

(57) ABSTRACT

A method for morphing a surgical object for a surgical navigation system is provided. The method comprises providing a tracking system and a surgical tool detectable by the tracking system. Dimensional data is collected and analyzed on the surgical object by contacting the surgical tool with the surgical object at more than one point while tracking the surgical tool with the tracking system, and the collected and analyzed dimensional data is associated with a reference model from a computer database of the tracking system. The reference model is selected and information for performing the surgery based upon the selected reference model is generated.

### RELATED APPLICATIONS

This application claims priority to U.S. provisional application Ser. No. 60/697,093, filed Jul. 7, 2005, the disclosure of which is expressly incorporated by reference herein.

### FIELD OF THE INVENTION

The present teachings relate to surgical navigation and more particularly to a method of using a surgical registration or characterization process to morph an instrument or implant with a surgical navigation system.

### BACKGROUND

Surgical navigation systems, also known as computer assisted surgery and image guided surgery, aid surgeons in locating patient anatomical structures, guiding surgical instruments, and implanting medical devices with a high degree of accuracy. Surgical navigation has been compared to a global positioning system that aids vehicle operators to navigate the earth. A surgical navigation system typically includes a computer, a tracking system, and patient anatomical information. The patient anatomical information can be obtained by using an imaging mode such as fluoroscopy, computer tomography (CT) or by simply defining the location of patient anatomy with the surgical navigation system. Surgical navigation systems can be used for a wide variety of surgeries to improve patient outcomes.

To successfully implant a medical device, surgical navigation systems often employ various forms of computing technology, as well as utilize intelligent instruments, digital touch devices, and advanced 3-D visualization software programs. All of these components enable surgeons to perform a wide variety of standard and minimally invasive surgical procedures and techniques. Moreover, these systems allow surgeons to more accurately plan, track and navigate the placement of instruments and implants relative to a patient's body, as well as conduct pre-operative and intra-operative body imaging.

Over time, surgeons often develop preferences for particular instruments and implant components that enable them to perform surgeries more efficiently and with significant benefits to their patients. Moreover, the availability of various computer assisted surgery navigation systems gives surgeons significant flexibility in selecting the surgical objects (implants or instruments) that, in their opinion and experience, are best suited for the particular surgery. While some surgical object manufacturers provide three-dimensional computer models corresponding to the various implants and instruments they sell, a number of implants and instruments remain for which a model has not been created or calibrated to work with a particular surgical instrument or with a specific surgical navigation system. Additionally, three-dimensional computer models of instruments and/or implants made for one navigation system might not always work with another system. Thus, it would be desirable to overcome these and other shortcomings of the prior art.

### SUMMARY OF THE INVENTION

The present teachings provide a method of using a registration or characterization process to morph an implant and/or instrument with a surgical navigation system.

In one exemplary embodiment, the present teachings provide a method for morphing a surgical object for a surgical navigation system. The method comprises providing a tracking system and a surgical tool detectable by the tracking system. The surgical object is contacted with the surgical tool at more than one point while tracking the surgical tool with the tracking system, thereby collecting and analyzing dimensional data on the surgical object. The collected and analyzed dimensional data is associated with a reference model from a computer database of the tracking system, and the reference model is selected and information for performing the surgery based upon the selected reference model is generated.

### BRIEF DESCRIPTION OF THE DRAWINGS

The above-mentioned aspects of the present teachings and the manner of obtaining them will become more apparent and the invention itself will be better understood by reference to the following description of the embodiments of the invention taken in conjunction with the accompanying drawings, wherein:

FIG. 1 is a perspective view of an exemplary operating room setup in a surgical navigation embodiment in accordance with the present teachings;

FIG. 2 is an exemplary block diagram of a surgical navigation system embodiment in accordance with the present teachings;

FIGS. 3 and 4 are exemplary computer display layout embodiments in accordance with the present teachings;

FIG. 5 is an exemplary surgical navigation kit embodiment in accordance with the present teachings;

FIG. 6 is a flowchart illustrating the operation of an exemplary surgical navigation system in accordance with the present teachings;

FIGS. 7 and 8 are flowcharts illustrating exemplary methods in accordance with the present teachings;

FIG. 9 is a perspective view of a physician registering points on a biomedical implant associated with a calibration device in accordance with the present teachings;

FIG. 10 is a perspective view illustrating a biomedical implant having points registered during an exemplary morphing process in accordance with the present teachings; and

FIG. 11 is a perspective view illustrating a biomedical instrument having points registered during an exemplary morphing process in accordance with the present teachings.

Corresponding reference characters indicate corresponding parts throughout the several views.

### DETAILED DESCRIPTION

The embodiments of the present teachings described below are not intended to be exhaustive or to limit the invention to the precise forms disclosed in the following detailed description. Rather, the embodiments are chosen and described so that others skilled in the art may appreciate and understand the principles and practices of the present teachings.

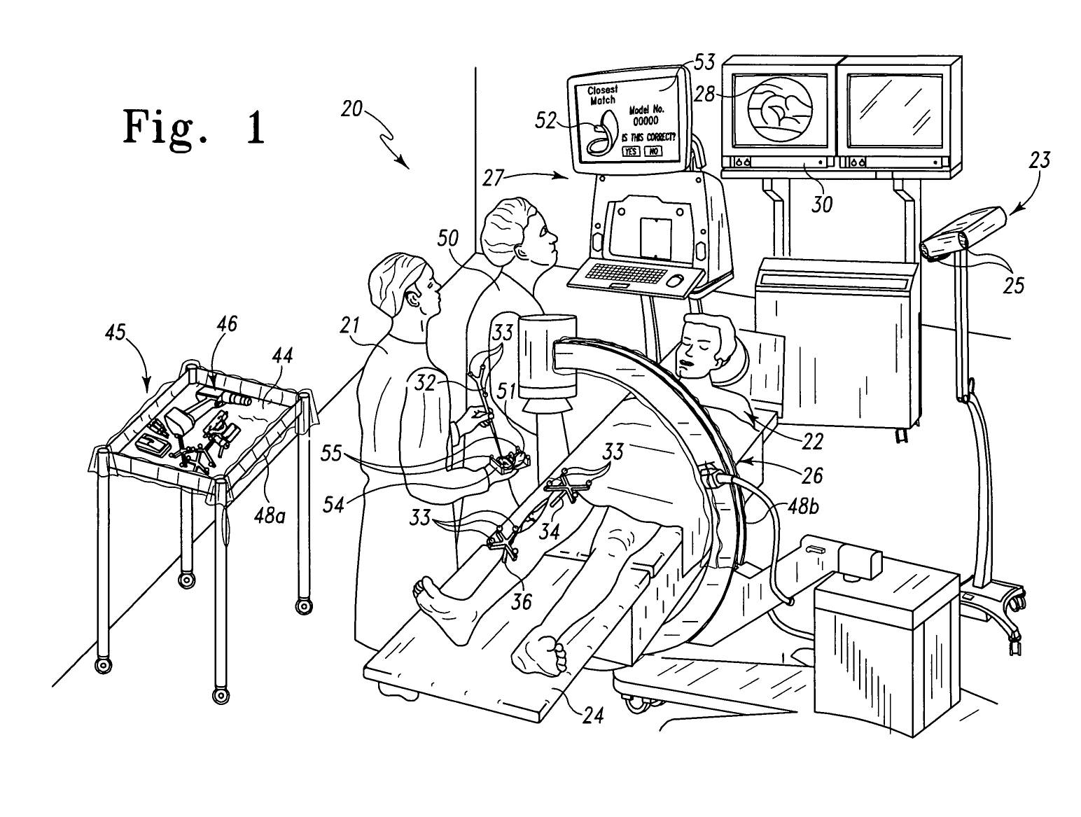

FIG. 1 shows a perspective view of an operating room with a surgical navigation system 20. Surgeon or physician 21 is aided by the surgical navigation system in performing knee arthroplasty, also known as knee replacement surgery, on patient 22 shown lying on operating table 24. Surgical navigation system 20 has a tracking system that locates trackers or arrays and tracks them in real-time. To accomplish this, the surgical navigation system includes optical locator 23, which has two CCD (charge couple device) cameras 25 that detect the positions of the trackers in space by using triangulation methods. The relative location of the trackers, including the patient's anatomy, can then be shown on a computer display (such as computer display 27 for instance) to assist the surgeon during the surgical procedure. The trackers that are typically used include probe trackers, instrument trackers, reference trackers, and calibrator trackers. The operating room includes an imaging system such as C-arm fluoroscope 26 with fluoroscope display image 28 to show a real-time image of the patient's knee on monitor 30. The tracking system also detects the location of surgical probe 32, as well as reference trackers or arrays 34, 36, which are attached to the patient's femur and tibia. By knowing the location of markers 33 attached to the surgical components, the tracking system can detect and calculate the position of the components in space. The operating room also includes instrument cart 45 having tray 44 for holding a variety of surgical instruments and trackers 46. Instrument cart 45 and C-arm 26 are typically draped in sterile covers 48a, 48b to eliminate contamination risks within the sterile field.

The surgery is performed within a sterile field, adhering to the principles of asepsis by all scrubbed persons in the operating room. Patient 22, surgeon 21 and assisting clinician 50 are prepared for the sterile field through appropriate scrubbing and clothing. The sterile field will typically extend from operating table 24 upward in the operating room. Typically both the computer display and fluoroscope display are located outside of the sterile field.

A representation of the patient's anatomy can be acquired with an imaging system, a virtual image, a morphed image, or a combination of imaging techniques. The imaging system can be any system capable of producing images that represent the patient's anatomy such as a fluoroscope producing x-ray two-dimensional images, computer tomography (CT) producing a three-dimensional image, magnetic resonance imaging (MRI) producing a three-dimensional image, ultrasound imaging producing a two-dimensional image, and the like. A virtual image of the patient's anatomy can be created by defining anatomical points with the surgical navigation system 20 or by applying a statistical anatomical model. A morphed image of the patient's anatomy can be created by combining an image of the patient's anatomy with a data set, such as a virtual image of the patient's anatomy. Some imaging systems, such as C-arm fluoroscope 26, can require calibration. The C-arm can be calibrated with a calibration grid that enables determination of fluoroscope projection parameters for different orientations of the C-arm to reduce distortion. A registration phantom can also be used with a C-arm to coordinate images with the surgical navigation application program and improve scaling through the registration of the C-arm with the surgical navigation system. A more detailed description of a C-arm based navigation system is provided in James B. Stiehl et al., Navigation and Robotics in Total Joint and Spine Surgery, Chapter 3: C-Arm-Based Navigation, Springer-Verlag (2004).

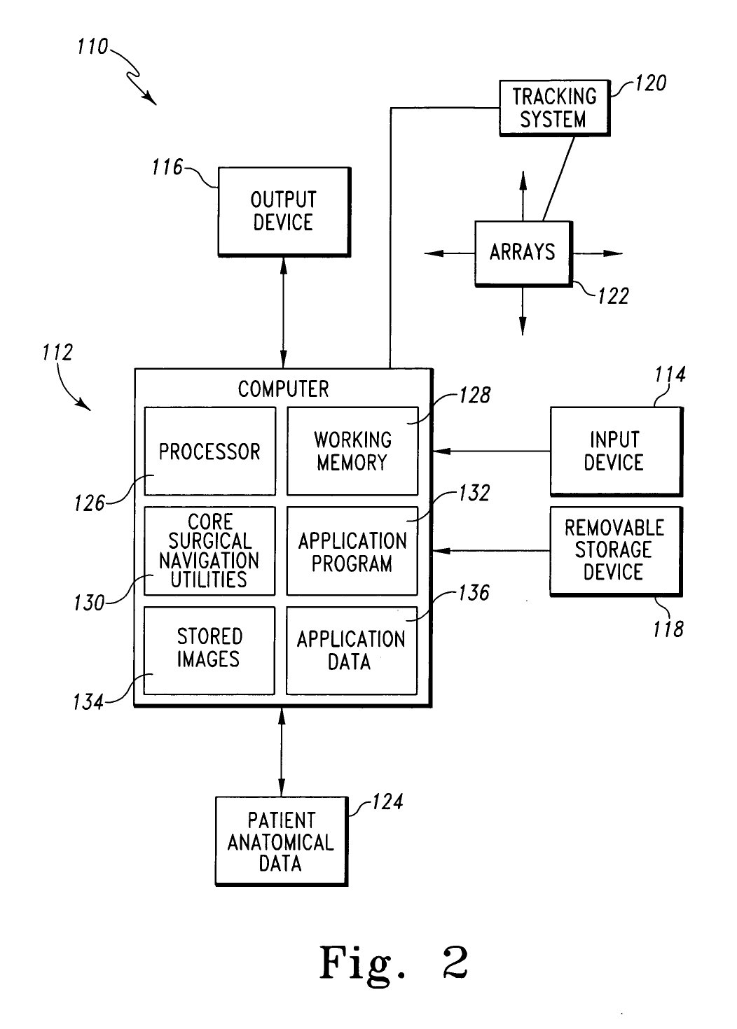

FIG. 2 is a block diagram of an exemplary surgical navigation system embodiment in accordance with the present teachings, such as the Acumen™ Surgical Navigation System, available from EBI, L.P., Parsippany, New Jersey USA, a Biomet Company. The surgical navigation system 110 comprises computer 112, input device 114, output device 116, removable storage device 118, tracking system 120, trackers or arrays 122, and patient anatomical data 124, as further described in the brochure Acumen™ Surgical Navigation System, Understanding Surgical Navigation (2003), available from EBI, L.P. The Acumen™ Surgical Navigation System can operate in a variety of imaging modes such as a fluoroscopy mode creating a two-dimensional x-ray image, a computer-tomography (CT) mode creating a three-dimensional image, and an imageless mode creating a virtual image or planes and axes by defining anatomical points of the patient's anatomy. In the imageless mode, a separate imaging device such as a C-arm is not required, thereby simplifying set-up. The Acumen™ Surgical Navigation System can run a variety of orthopedic applications, including applications for knee arthroplasty, hip arthroplasty, spine surgery, and trauma surgery, as further described in the brochure “Acumen™ Surgical Navigation System, Surgical Navigation Applications” (2003), available from EBI, L.P. A more detailed description of an exemplary surgical navigation system is provided in James B. Stiehl et al., Navigation and Robotics in Total Joint and Spine Surgery, Chapter 1: Basics of Computer-Assisted Orthopedic Surgery (CAOS), Springer-Verlag (2004).

Computer 112 can be any computer capable of properly operating surgical navigation devices and software, such as a computer similar to a commercially available personal computer that comprises a processor 126, working memory 128, core surgical navigation utilities 130, an application program 132, stored images 134, and application data 136. Processor 126 is a processor of sufficient power for computer 112 to perform desired functions, such as one or more microprocessors. Working memory 128 is memory sufficient for computer 112 to perform desired functions such as solid-state memory, random-access memory, and the like. Core surgical navigation utilities 130 are the basic operating programs, and include image registration, image acquisition, location algorithms, orientation algorithms, virtual keypad, diagnostics, and the like. Application program 132 can be any program configured for a specific surgical navigation purpose, such as orthopedic application programs for unicondylar knee (“uni-kee”), total knee, hip, spine, trauma, intramedullary (“IM”) nail, and external fixator. Stored images 134 are those recorded during image acquisition using any of the imaging systems previously discussed. Application data 136 is data that is generated or used by application program 132, such as implant geometries, instrument geometries, surgical defaults, patient landmarks, and the like. Application data 136 can be pre-loaded in the software or input by the user during a surgical navigation procedure.

Output device 116 can be any device capable of creating an output useful for surgery, such as visual or auditory output devices. Visual output devices can be any device capable of creating a visual output useful for surgery, such as a two-dimensional image, a three-dimensional image, a holographic image, and the like. The visual output device can be a monitor for producing two and three-dimensional images, a projector for producing two and three-dimensional images, and indicator lights. Auditory output devices can be any device capable of creating an auditory output used for surgery, such as a speaker that can be used to provide a voice or tone output.

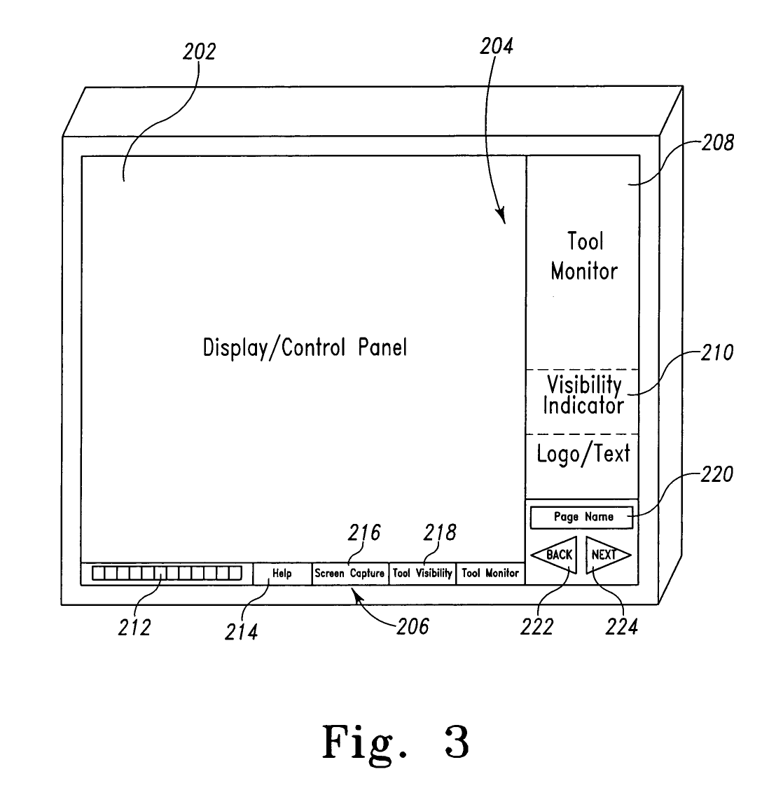

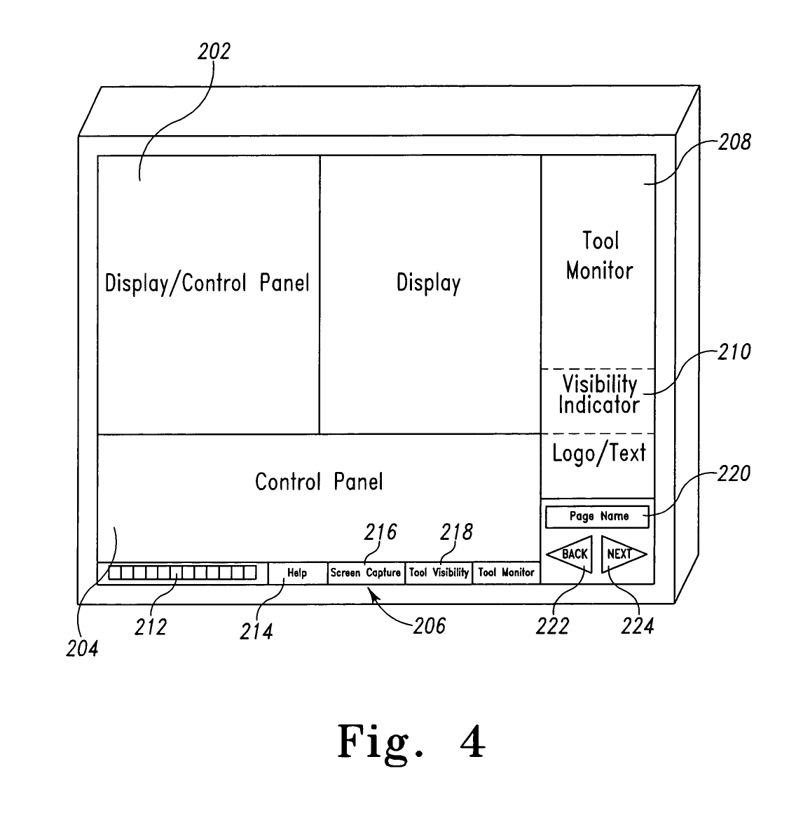

FIG. 3 shows a first computer display layout embodiment, and FIG. 4 shows a second computer display layout embodiment in accordance with the present teachings. The display layouts can be used as a guide to create common display topography for use with various embodiments of input devices 114 and to produce visual outputs for core surgical navigation utilities 130, application programs 132, stored images 134, and application data 136 embodiments. Each application program 132 is typically arranged into sequential pages of surgical protocol that are configured according to a graphic user interface scheme. The graphic user interface can be configured with a main display 202, main control panel 204, and tool bar 206. Main display 202 presents images such as selection buttons, image viewers, and the like. Main control panel 204 can be configured to provide information such as tool monitor 208, visibility indicator 210, and the like. Tool bar 206 can be configured with a status indicator 212, help button 214, screen capture button 216, tool visibility button 218, current page button 220, back button 222, forward button 224, and the like. Status indicator 212 provides a visual indication that a task has been completed, visual indication that a task must be completed, and the like. Help button 214 initiates a pop-up window containing page instructions. Screen capture button 216 initiates a screen capture of the current page and the tracked elements will be displayed when the screen capture is taken. Tool visibility button 218 initiates a visibility indicator pop-up window or adds a tri-planar tool monitor to control panel 204 above current page button 220. Current page button 220 can display the name of the current page and initiate a jump-to menu when pressed. Forward button 224 advances the application to the next page. Back button 222 returns the application to the previous page. The content in the pop-up will be different for each page.

Referring again to FIG. 2, removable storage device 118 can be any device having a removable storage media that would allow downloading data such as application data 136 and patient anatomical data 124. The removable storage device can be a read-write compact disc (CD) drive, a read-write digital video disc (DVD) drive, a flash solid-state memory port, a removable hard drive, a floppy disc drive, and the like.

Tracking system 120 can be any system that can determine the three-dimensional location of devices carrying or incorporating markers that serve as tracking indicia. An active tracking system has a collection of infrared light emitting diode (ILEDs) illuminators that surround the position sensor lenses to flood a measurement field of view with infrared light. A passive system incorporates retro-reflective markers that reflect infrared light back to the position sensor, and the system triangulates the real-time position (x, y, and z location) and orientation (rotation around x, y, and z axes) of a tracker or array 122 and reports the result to the computer system with an accuracy of about 0.35 mm Root Mean Squared (RMS). An example of a passive tracking system is a Polaris® Passive System and an example of a marker is the NDI Passive Spheres™, both available from Northern Digital Inc. Ontario, Canada. A hybrid tracking system can detect active and active wireless markers in addition to passive markers. Active marker based instruments enable automatic tool identification, program control of visible LEDs, and input via tool buttons. An example of a hybrid tracking system is the Polaris® Hybrid System, available from Northern Digital Inc. A marker can be a passive IR reflector, an active IR emitter, an electromagnetic marker, and an optical marker used with an optical camera.

As is generally known within the art, implants and instruments may also be tracked by electromagnetic tracking systems. These systems locate and track devices and produce a real-time, three-dimensional video display of the surgical procedure. This is accomplished by using electromagnetic field transmitters that generate a local magnetic field around the patient's anatomy. In turn, the localization system includes magnetic sensors that identify the position of tracked instruments as they move relative to the patient's anatomy. By not requiring a line of sight with the transmitter, electromagnetic systems are also adapted for in vivo use, and are also integrable, for instance, with ultrasound and CT imaging processes for performing interventional procedures by incorporating miniaturized tracking sensors into surgical instruments. By processing transmitted signals generated by the tracking sensors, the system is able to determine the position of the surgical instruments in space, as well as superimpose their relative positions onto pre-operatively captured CT images of the patient.

Trackers or arrays 122 can be probe trackers, instrument trackers, reference trackers, calibrator trackers, and the like. Trackers 122 can have any number of markers, but typically have three or more markers to define real-time position (x, y, and z location) and orientation (rotation around x, y, and z axes). As will be explained in greater detail below, a tracker comprises a body and markers. The body comprises an area for spatial separation of markers. In some embodiments, there are at least two arms and some embodiments can have three arms, four arms, or more. The arms are typically arranged asymmetrically to facilitate specific tracker and marker identification by the tracking system. In other embodiments, such as a calibrator tracker, the body provides sufficient area for spatial separation of markers without the need for arms. Trackers can be disposable or non-disposable. Disposable trackers are typically manufactured from plastic and include installed markers. Non-disposable trackers are manufactured from a material that can be sterilized, such as aluminum, stainless steel, and the like. The markers are removable, so they can be removed before sterilization.

Planning and collecting patient anatomical data 124 is a process by which a clinician inputs into the surgical navigation system actual or approximate anatomical data. Anatomical data can be obtained through techniques such as anatomic painting, bone morphing, CT data input, and other inputs, such as ultrasound and fluoroscope and other imaging systems.

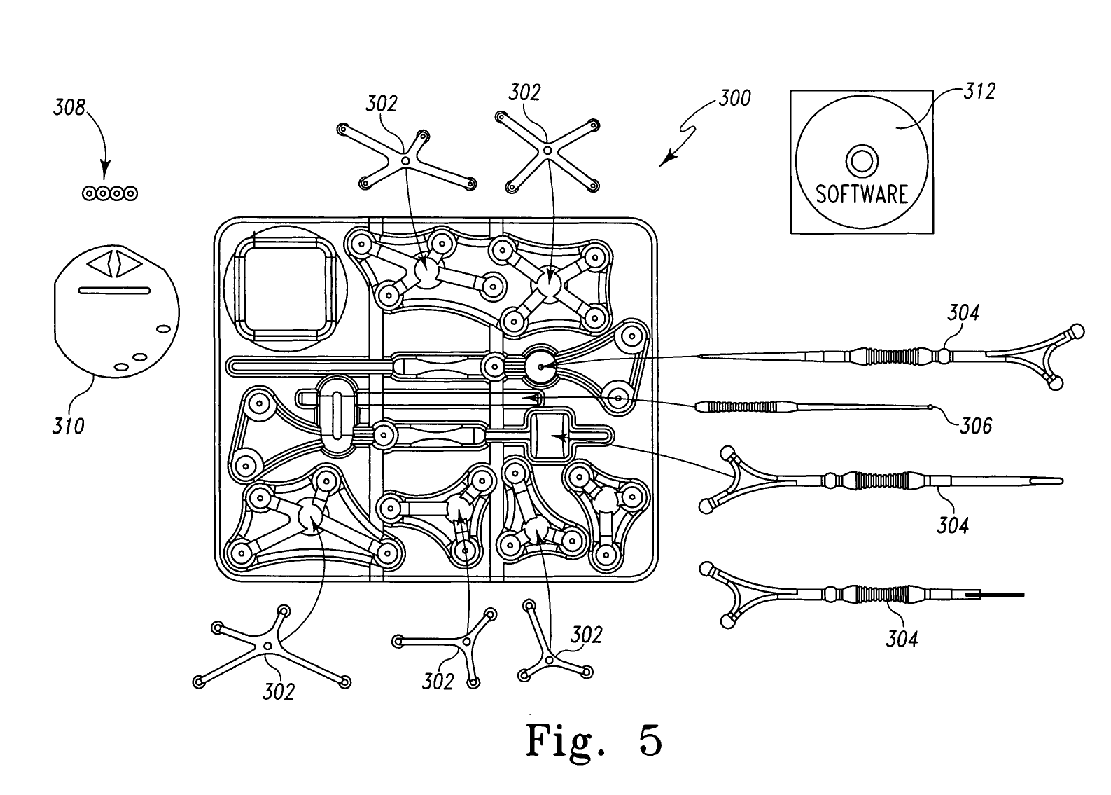

FIG. 5 shows orthopedic application kit 300, which is used in accordance with the present teachings. Application kit 300 is typically carried in a sterile bubble pack and is configured for a specific surgery. Exemplary kits can comprise one or more trackers or arrays 302, surgical probes 304, stylus 306, markers 308, virtual keypad template 310, and application program 312. Orthopedic application kits are available for unicondylar knee, total knee, total hip, spine, and external fixation from EBI, L.P.

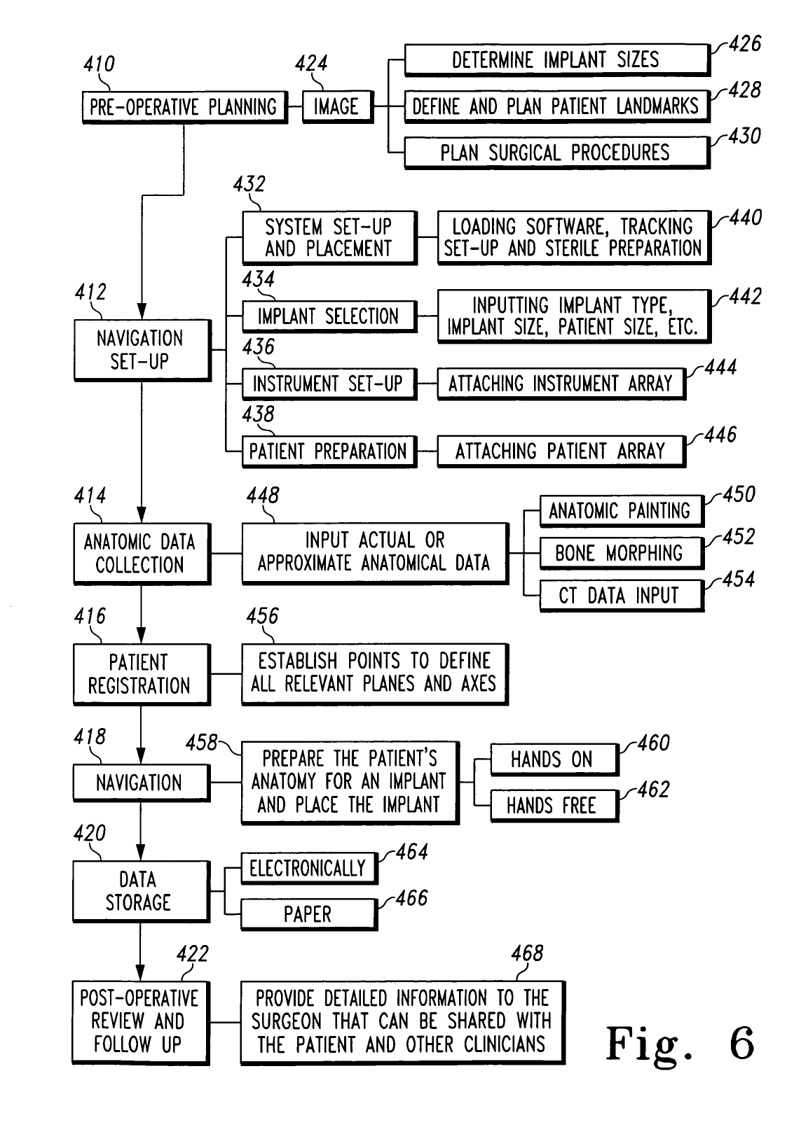

FIG. 6 shows an operational flowchart of a surgical navigation system in accordance with the present teachings. The process of surgical navigation can include the elements of pre-operative planning 410, navigation set-up 412, anatomic data collection 414, patient registration 416, navigation 418, data storage 420, and post-operative review and follow-up 422.

Pre-operative planning 410 is performed by generating an image 424, such as a CT scan that is imported into the computer. With image 424 of the patient's anatomy, the surgeon can then determine implant sizes 426, such as screw lengths, define and plan patient landmarks 428, such as long leg mechanical axis, and plan surgical procedures 430, such as bone resections and the like. Pre-operative planning 410 can reduce the length of intra-operative planning thus reducing overall operating room time.

Navigation set-up 412 includes the tasks of system set-up and placement 432, implant selection 434, instrument set-up 436, and patient preparation 438. System set-up and placement 432 includes loading software, tracking set-up, and sterile preparation 440. Software can be loaded from a pre-installed application residing in memory, a single use software disk, or from a remote location using connectivity such as the internet. A single use software disk contains an application that will be used for a specific patient and procedure that can be configured to time-out and become inoperative after a period of time to reduce the risk that the single use software will be used for someone other than the intended patient. The single use software disk can store information that is specific to a patient and procedure that can be reviewed at a later time. Tracking set-up involves connecting all cords and placement of the computer, camera, and imaging device in the operating room. Sterile preparation involves placing sterile plastic on selected parts of the surgical navigation system and imaging equipment just before the equipment is moved into a sterile environment, so the equipment can be used in the sterile field without contaminating the sterile field.

Navigation set-up 412 is completed with implant selection 434, instrument set-up 436, and patient preparation 438. Implant selection 434 involves inputting into the system information such as implant type, implant size, patient size, operative side and the like 442. Instrument set-up 436 involves attaching an instrument tracker to each instrument intended to be used and then calibrating each instrument 444. Instrument trackers should be placed on instruments, so the instrument tracker can be acquired by the tracking system during the procedure. Patient preparation 438 is similar to instrument set-up because a tracker is typically rigidly attached to the patient's anatomy 446. Reference trackers do not require calibration but should be positioned so the reference tracker can be acquired by the tracking system during the procedure.

As mentioned above, anatomic data collection 414 involves a clinician inputting into the surgical navigation system actual or approximate anatomical data 448. Anatomical data can be obtained through techniques such as anatomic painting 450, bone morphing 452, CT data input 454, and other inputs, such as ultrasound and fluoroscope and other imaging systems. The navigation system can construct a bone model with the input data. The model can be a three-dimensional model or two-dimensional pictures that are coordinated in a three-dimensional space. Anatomical painting 450 allows a surgeon to collect multiple points in different areas of the exposed anatomy. The navigation system can use the set of points to construct an approximate three-dimensional model of the bone. The navigation system can use a CT scan done pre-operatively to construct an actual model of the bone. Fluoroscopy uses two-dimensional images of the actual bone that are coordinated in a three-dimensional space. The coordination allows the navigation system to accurately display the location of an instrument that is being tracked in two separate views. Image coordination is accomplished through a registration phantom that is placed on the image intensifier of the C-arm during the acquisition of images. The registration phantom is a tracked device that contains imbedded radio-opaque spheres. The spheres have varying diameters and reside on two separate planes. When an image is taken, the fluoroscope transfers the image to the navigation system. Included in each image are the imbedded spheres. Based on previous calibration, the navigation system is able to coordinate related anterior and posterior views and coordinate related medial and lateral views. The navigation system can also compensate for scaling differences in the images.

Patient registration 416 establishes points that are used by the navigation system to define all relevant planes and axes 456. Patient registration 416 can be performed by using a probe tracker to acquire points, placing a software marker on a stored image, or automatically by software identifying anatomical structures on an image or cloud of points. Once registration is complete, the surgeon can identify the position of tracked instruments relative to tracked bones during the surgery. The navigation system enables a surgeon to interactively reposition tracked instruments to match planned positions and trajectories and assists the surgeon in navigating the patient's anatomy.

During the procedure, step-by-step instructions for performing the surgery in the application program are provided by a navigation process. Navigation 418 is the process a surgeon uses in conjunction with a tracked instrument or other tracked array to precisely prepare the patient's anatomy for an implant and to place the implant 458. Navigation 418 can be performed hands-on 460 or hands-free 462. However navigation 418 is performed, there is usually some form of feedback provided to the clinician such as audio feedback or visual feedback or a combination of feedback forms. Positive feedback can be provided in instances such as when a desired point is reached, and negative feedback can be provided in instances such as when a surgeon has moved outside a predetermined parameter. Hands-free 462 navigation involves manipulating the software through gesture control, tool recognition, virtual keypad and the like. Hands-free 462 is done to avoid leaving the sterile field, so it may not be necessary to assign a clinician to operate the computer outside the sterile field.

Data storage 420 can be performed electronically 464 or on paper 466, so information used and developed during the process of surgical navigation can be stored. The stored information can be used for a wide variety of purposes such as monitoring patient recovery and potentially for future patient revisions. The stored data can also be used by institutions performing clinical studies.

Post-operative review and follow-up 422 is typically the final stage in a procedure. As it relates to navigation, the surgeon now has detailed information that he can share with the patient or other clinicians 468.

The present teachings enhance surgical navigation system 20 by incorporating into the system a registration process for morphing a surgical object or component (e.g., biomedical implant or instrument). More particularly, in addition to tracking surgical components, the navigation system also registers or characterizes the dimensional data or physical parameters that define these surgical components and incorporates this data into the navigation system so that these components can be used during a surgical procedure. System 20 can also determine or suggest appropriate surgical information (e.g., surgical planning, anatomical resections, sizing and rotational data, such as anteversion, medialization, inclination and lateralization, length and depth information and/or necessary adjustments to external instruments, jigs and fixturing devices) needed to perform the surgical procedure in light of this object's use.

As shown in FIG. 1, surgeon 21 performs a morphing procedure in which he creates a virtual object of surgical device 51 (depicted here as a knee implant) by touching surgical probe 32 against surgical device 51 at points along its surface. More particularly, one or more points along the surface of the device are registered or characterized by the surgeon and collected by system 20. To recognize and collect the spatial position coordinates of probe 32 as it registers one or more selected points along the surface of surgical device 51, the surgical device must either remain static or stay in a fixed location relative to an object that is detectable by the tracking system. More particularly, cameras 25 must be able to detect and triangulate the spatial position of surgical device 51 as the surgical tool or probe is registered with its surface in order to generate a virtual object of the device. To accomplish this, surgical device 51 is coupled to calibration device 54, which has calibrator markers 55 detectable by the tracking system attached thereto. To couple the surgical device to the calibrator, the surgical device is fitted into internal grooves or slots (not shown) contained on the internal walls or sides of the calibration device. Alternatively, the calibration device can be equipped with a locking strap or other such locking means for holding the surgical device into place. Because of the fixed relationship between calibration device 54 and surgical device 51, the tracking system is able to detect and calculate the position of the surgical device in space by tracking the position of the calibrator markers. In addition to calibration devices, it should also be appreciated that instrument tracking structures can alternatively be coupled to the surgical devices for determining their spatial positions with the tracking system. A more detailed description of these instrument trackers is provided above.

The points to be registered can be chosen randomly or specifically selected such that one or more unique features essential to the operation of the device are identified. However the points are chosen, once the surgeon registers probe 32 against a sufficient number of points along the surface of surgical device 51, software associated with the surgical navigation system analyzes the relative locations of the points collected and generates virtual object 52 of surgical device 51 and displays it on monitor 53 of computer display 27. That is, virtual object 52 is created by acquiring the spatial position coordinates corresponding to a plurality of points on the surface of surgical device 51, and subsequently mapping the spatial position coordinates to create a digital model of the surgical object. Virtual object 52 can be either three-dimensional or two-dimensional and can be used by the navigation system to guide a surgeon during a surgical procedure, as well as by a surgical simulation program. A more detailed description of an exemplary surgical morphing process is provided in James B. Stiehl et al., Navigation and Robotics in Total Joint and Spine Surgery, Chapter 5: Bone Morphing: 3D Reconstruction Without Pre- or Intraoperative Imaging-Concept and Applications, Springer-Verlag (2004).

Once virtual object 52 has been created, it is associated with one or more reference models contained within a computer database associated with the surgical navigation tracking system. More particularly, the computer database retrieves, and the monitor displays, one or more reference models that closely resemble the shape ascribed to virtual object 52. The surgeon then selects the reference model that most closely resembles virtual object 52. After a reference model has been selected, its dimensions are modified or morphed to identically match the dimensions of virtual object 52, and the modified dimensional data is saved in the computer database. Moreover, once the reference model has been selected and identified, software associated with the navigation system can also automatically retrieve and load specific surgical instructions pertaining to a procedure involving such model or specific preferences used by a particular user.

As explained in detail below, the steps associated with the present morphing process may be conducted in various chronological orders. For instance, a surgical object may be first registered and then compared to a generically shaped reference model stored within the system's computer database. The computer generated reference model is then modified or morphed to match the actual dimensional parameters of the registered device. Alternatively, the computer generated reference model may be selected from the computer database prior to registering the surgical device. In this case, the physician selects the computer generated reference model that most closely resembles the surgical object to be characterized and then performs the registration process on the object. Thereafter, the dimensional parameters of the computer generated reference model are morphed to match the actual dimensions of the physical device to be implanted. According to this illustrated embodiment, software associated with the navigation system automatically alters the dimensional parameters of the computer generated reference model and stores it in the system's database. In other alternative methods, the dimensional parameters of the altered reference model are manually entered into the database after the dimensional data of the physical device is collected and analyzed through the registration process. As such, the present teachings are not intended to be limited and thereby contemplate a wide variety of means for registering and morphing surgical devices.

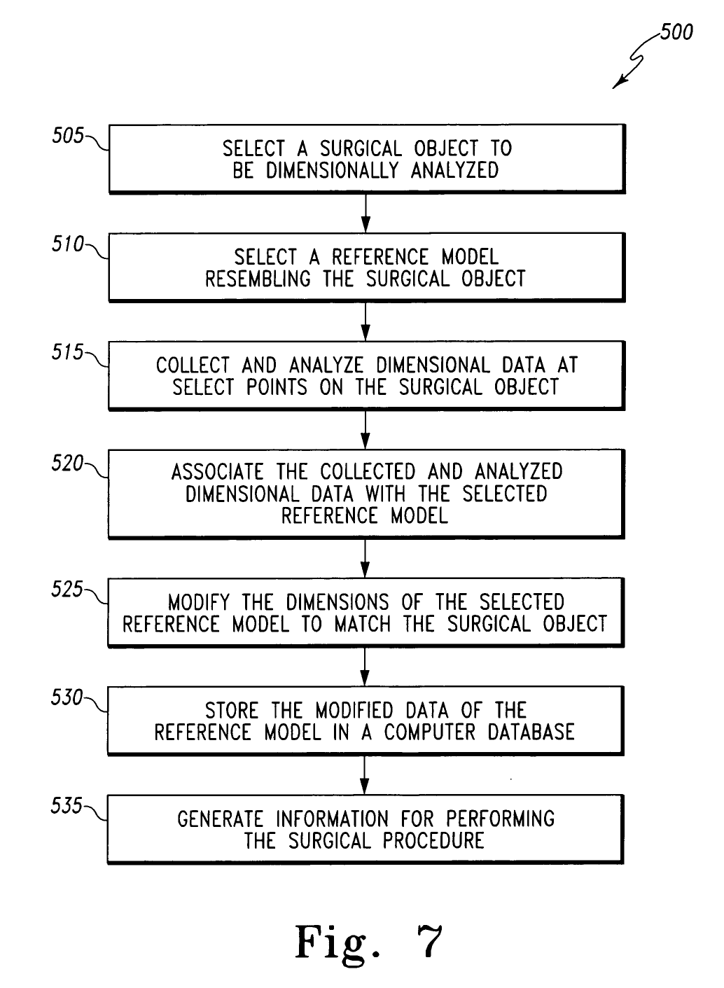

One exemplary registration and morphing process 500 in accordance with the present teachings is shown in FIG. 7. Surgeon 21 first selects a surgical object (such as surgical device 51 in FIG. 1) to be dimensionally characterized or analyzed (step 505). For instance, if the surgical navigation procedure requires implanting a prosthetic knee component, the surgeon selects the actual knee implant to be surgically implanted as the device to be analyzed. The surgeon then selects a reference model from the navigation system's computer database that closely resembles the surgical object to be dimensionally analyzed (step 510). For instance, in a knee arthroplasty involving a knee prosthetic, the surgeon will browse the computer database for all knee prosthetic components stored within the database. Upon identifying the knee model that most closely resembles that actual knee component to be surgically implanted, the surgeon will select this model as the reference model.

Next, surgeon 21 registers or touches probe 32 at various points along the surface of the surgical object to collect and analyze dimensional data along the surface of the surgical object (step 515). The surgeon then associates the collected and analyzed dimensional data with the selected computer generated reference model (step 520). After surgeon 21 collects data at several points along the surface of the surgical object, the dimensions of the selected reference model are modified or morphed to identically match the dimensional data of the surgical object (step 525). After the dimensions of the selected reference model are morphed to match the surgical object, the surgeon stores the modified data of the reference model in the computer database (step 530) so that the information may be subsequently accessed as needed to assist in conducting further surgical navigation procedures. Moreover, once the surgical object has been registered and matched to a reference model stored within the computer database, the system generates information for planning and performing the surgical procedure (step 535). For instance, by knowing the dimensions of the surgical component, the system is able to determine appropriate anatomical resections and provide relative resection information for adjusting external instruments, jigs and fixtures that are used during the surgical procedure.

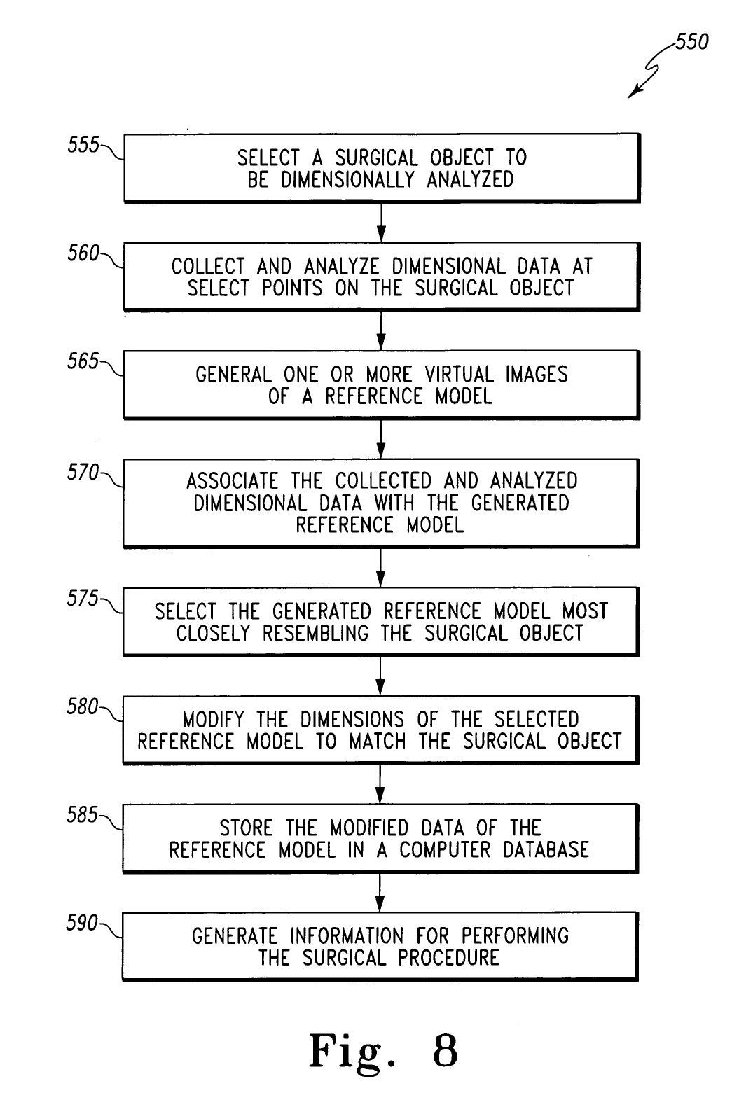

Another illustration of a morphing process (550) is depicted in FIG. 8. Surgeon 21 first selects a surgical object (such as surgical device 51 in FIG. 1) to be dimensionally analyzed (step 555). Next, surgeon 21 registers or touches probe 32 at various points along the surface of the surgical object to collect and analyze dimensional data of the surgical object (step 560). After surgeon 21 collects data at several points along the surface of the surgical object, the computer database generates one or more virtual images of a reference model closely resembling the dimensional parameters of the surgical object (step 565). The surgeon or system next associates the collected and analyzed dimensional data with the generated reference model (step 570). Surgeon 21 or the system next selects the generated reference model which most closely resembles the dimensional parameters of the surgical object (step 575). Surgeon 21 or the system then modifies the dimensions of the selected reference model to identically match the dimensional data of the surgical object (step 580) and then stores the modified data of the reference model in the computer database (step 585). Finally, once the surgical object has been matched to a reference model and the reference model morphed to identically match the actual surgical object being registered, the morphed reference model is stored within the computer database and the system generates information for performing the surgical procedure (step 590) based upon this stored information.

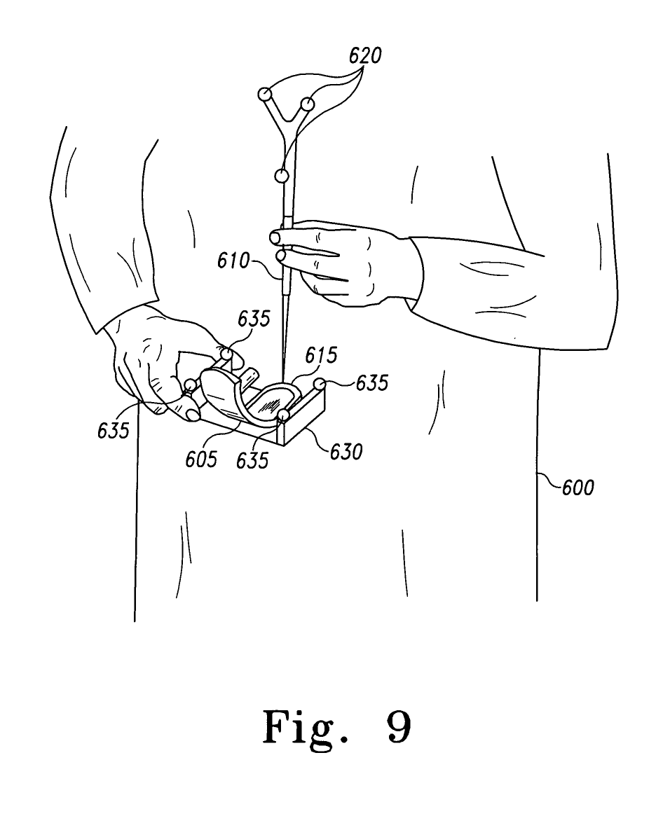

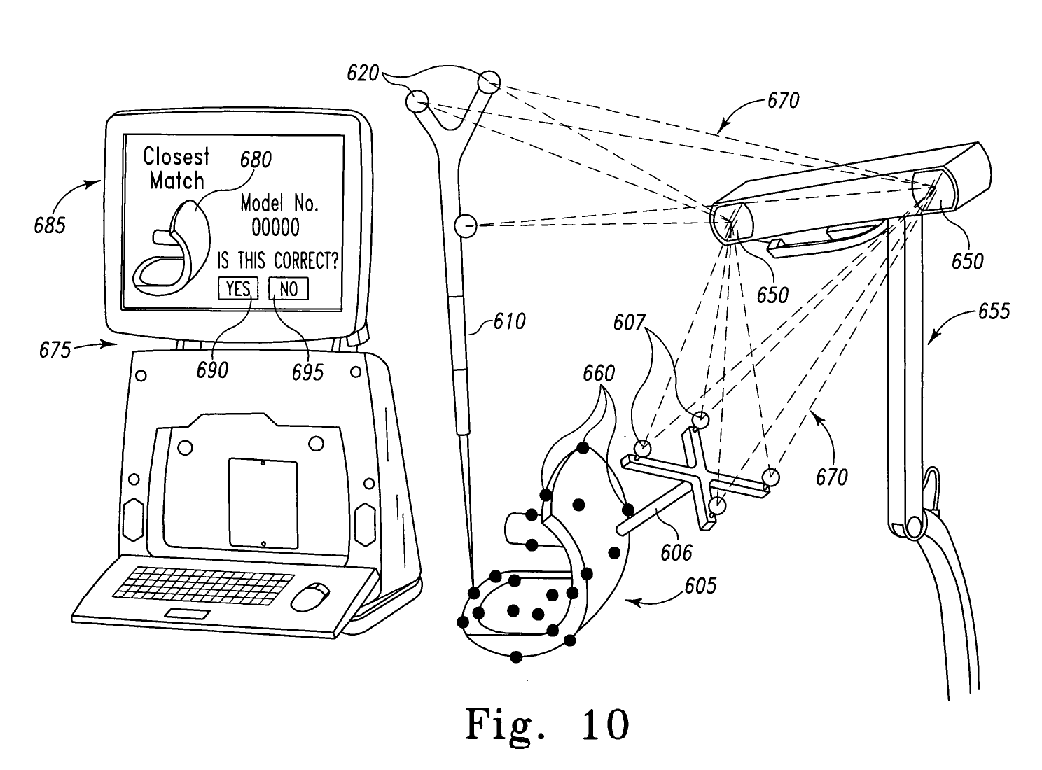

An illustration of a biomedical implant undergoing a morphing process in accordance with the present teachings is depicted in FIG. 9. Surgeon 600 registers several points along surface 615 of implant 605 (illustrated here as a knee implant) by touching the tip of probe 610 against the surface. As probe 610 registers the plurality of select points along surface 615 of implant 605, cameras 650 of optical locator 655 (see FIG. 10) detect the positions of markers 620 on probe 610 and calibration device 630 (having calibration markers 635 affixed thereto) to triangulate and analyze the relative spatial position coordinates that correspond to the plurality of select points along surface 615 of the implant. This process is accomplished by using algorithms, such as the direct linear transform (DLT) process, which reconstructs 3D coordinates of each of the tracked markers 620, 635.

Another exemplary illustration of implant 605 undergoing a morphing process is depicted in FIG. 10. Surgeon 600 touches or registers the tip of probe 610 against implant 605 at a plurality of select points 660 (shown as black dots on the surface of the implant) along its surface to collect and analyze dimensional data of the surgical implant 605. As probe 610 touches the plurality of select points 660, cameras 650 of optical locator 655 detect the positions of markers 620 on probe 610 and markers 607 of detachable instrument tracker 606 (see the optical path/measurement field of the tracking system represented by dashed lines 670) and collect and analyze the relative spatial position coordinates that correspond to the plurality of select points 660 along the surface of implant 605. This process is accomplished by using algorithms to reconstruct 3D coordinates of each of the detected markers 620, 607.

Once the system calculates the dimensional parameters of implant 605, the data is analyzed by software contained on computer system 675. A virtual image 680 of one or more reference implant models stored on a computer database and dimensionally resembling implant 605 is then generated and displayed on computer monitor 685. Surgeon 600 is then prompted to select whether the generated virtual image 680 is correct or not (i.e., whether the generated implant is dimensionally similar to implant 605). If the suggested implant match is correct, the surgeon can select the “yes” button 690 on monitor 685, whereby the software then generates information for performing a surgical procedure with implant 605. Alternatively, if the suggested implant match is incorrect (i.e., the suggested implant is not dimensionally similar to implant 605), the surgeon can select the “no” button 695 on monitor 685, and the surgeon is either prompted to select another close match or manually enter or record the dimensional surface data into the database to be stored as a new implant entry.

As explained above, it should be appreciated that the order the morphing steps take place may be modified as needed. For instance, the surgeon may decide to first access the computer database and then register or characterize a surgical object to determine whether a generically shaped reference model resembling the object can be located within the database. If the surgeon locates a closely matching reference model, the surgeon can then be prompted to select this model and use it as a template while the surgical object is registered with the surgical probe. More particularly, once the reference model is selected, the surgeon is prompted to identify select points along the surface of the surgical object in a manner such that the reference surgical model is automatically altered/modified to match the dimensional parameters of the surgical object being registered. As optical locator 655 detects and triangulates the positions of markers 620 on probe 610 and markers 607 on detachable instrument tracker 606 corresponding to a plurality of select points 660 along the surface of implant 605, the software alters the dimensions of the reference model and reconstructs 3D coordinates of each of tracked markers 620, 607 in space.

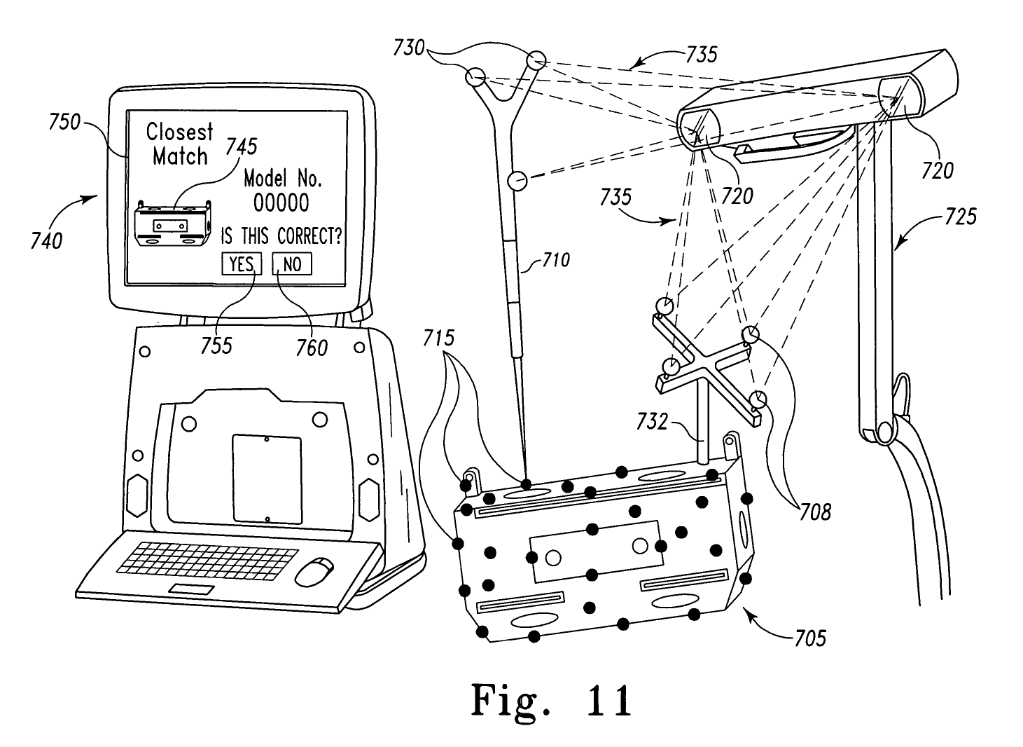

In addition to morphing biomedical implants as explained above, biomedical instruments may also be morphed. With reference to FIG. 11, an instrument morphing process is depicted in which data is collected and analyzed along the surface of instrument 705 (shown here as a cutting block) by touching or registering probe 710 against instrument 705 at a plurality of select surface points 715 (shown as black dots on the surface of the instrument). As probe 710 touches the plurality of select points 715 along the surface of instrument 705, cameras 720 of optical locator 725 detect and triangulate the positions of markers 730 on probe 710 and markers 708 on detachable instrument tracker 732 (see the optical path/measurement field represented by dashed lines 735) and analyze the relative spatial position coordinates that correspond to the plurality of select surface points 715 along the surface of instrument 705. This is done with algorithms that reconstruct 3D coordinates of each of the detected markers 730, 708.

Once the system calculates the dimensional parameters of instrument 705, the data is analyzed by software stored on computer system 740. The software generates virtual image 745 on monitor 750 of one or more reference instrument models that are stored within a computer database that closely resemble the dimensional parameters of instrument 705. Surgeon 600 may then be prompted to select whether the closest matching reference instrument model found on the system is correct or not (i.e., whether the suggested reference instrument is similar to the dimensional parameters of instrument 705). For instance, if the registered instrument and its associated dimensional information are already stored in the database, the software may then prompt surgeon 600 to verify that the matching reference instrument model is in fact the exact instrument the surgeon is morphing. If the suggested instrument match is correct, the surgeon can select the “yes” button 755 on monitor 750, at which time the software provides any known surgical information pertaining to a surgical procedure involving instrument 705. Alternatively, if the suggested instrument generated by the software is incorrect (i.e., the suggested reference instrument does not match instrument 705), the surgeon can select the “no” button 760 on monitor 750, and the surgeon is either prompted to select another close match or manually enters or records the dimensional surface data into the database to be stored as a new instrument entry.

Advantages and improvements of the methods of the present invention are demonstrated in the following examples. The examples are illustrative only and are not intended to limit or preclude other embodiments of the invention.

According to one exemplary example, a femoral component is analyzed to determine various resection planes (chamfer, interior and posterior cuts) and gap analysis information by registering several points of the femoral component with a surgical probe. To determine these cuts, a surgical probe registers critical axes and points along the inner profile or surface of the implant, such as the axis that runs perpendicularly to the intersection point at which the two angle cuts or planes of the femoral component come together. In other words, if one were looking at the implant from a lateral or side view, the probe would trace the axis that goes into the page where the planes defining the angle cuts of the implant intersect at the crotch of the component. By having the dimensional relationship of this axis as it is defined along the inner profile of the component, the plane perpendicular to this axis can then be added to the computer generated image to thereby create a three-dimensional representation of the implant. This information can then be used to fine adjust a cutting block, for instance, interiorly/posteriorly and/or medially/laterally on the distal femur for performing the final cuts before attaching the cutting block to the bone.

In addition to the inner profile, the outer profile (defining the three-dimensional curved shape of the implant's exterior surface) is also analyzed to determine information on gap analysis (e.g., to balance the compartmental gaps of a knee replacement procedure), as well as to determine the thickness of an implant and/or the distance between two implants once installed. The probe registers various points along the outer surface of the implant with the probe to define the three-dimensional shape of the component. This information is then input into the computer system to determine how much bone must be resected from the distal and posterior condyles to match the anatomy of the bone.

According to another exemplary example, a cutting block is characterized to determine resection and fixation information needed to attach the block to the patient's femur during a knee surgery. To characterize the cutting block, the system prompts the surgeon to identify with the probe essential features of the block, such as the cutting slot and pin holes. By acquiring this information, the system is able to determine how the cutting block must be positioned and affixed to the femur so that it physically corresponds to a preplanned surgical resection plane. To correctly position the cutting block to the femur, a tracked drill guide is used to place guide pins into the bone at locations which correspond to the pin holes of the cutting block. In other words, the guide pins are placed such that the pins will physically align with the cutting block's pin holes when affixing the cutting block to the femur. Once the guide pins have been placed into the bone and the cutting block fitted accordingly, the block's cutting slot is positioned such that it aligns with the preplanned surgical resection plane shown on the surgical plan image. As such, the surgeon is able to accurately perform necessary resections (e.g., chamfer, interior and posterior) prior to fitting the implant on the bone. For a further description of a process and apparatus for positioning a surgical instrument, see U.S. Pat. No. 6,377,839 titled “Tool Guide for a Surgical Tool,” filed May 29, 1998, which is incorporated by reference herein in its entirety.

The present teachings also allow the verification of surgical information and the recalibration of instruments, implants and tools to ensure that surgical components are properly aligned and positioned during an implantation procedure. For instance, if a surgeon is placing an acetabular cup into the acetabulum, medialization is very important, as well as anteversion and inclination, particularly as the surgeon does not want to over medialize the implant into the pelvis. If this happens, the pelvic wall may rupture and/or internal organs may be damaged. Anteversion and inclination of the implant is important for optimizing range of motion and restoring proper leg alignment. To ensure proper outcomes, accurate information pertaining to the surgical procedure must be available to the surgeon. According to one exemplary embodiment, surgical information pertaining to an instrument's tip and axis is obtained by characterizing and digitizing the instrument according to the present teachings. For instance, the surgeon can take a tracked surgical probe and characterize the instrument by registering the probe against its surface at select points as described in detail above. As the surgical instrument is characterized, the navigation system is able to interpolate these values into pertinent axis information by considering the instrument's midpoints, centerlines etc. After determining the relevant axis information, the surgeon can then touch the probe against its distal end, for instance, and calibrate the instrument “on the fly” rather than through a traditional recalibration process. Additionally, the surgeon can remain on the same navigation page without sequencing back into a special calibration page.

While exemplary embodiments incorporating the principles of the present teachings have been disclosed hereinabove, the present teachings are not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A method for morphing a surgical object for a surgical navigation system, comprising:
(a) providing a tracking system and a surgical tool detectable by the tracking system;
(b) contacting the surgical tool at more than one point on the surgical object while tracking the surgical tool with the tracking system, thereby collecting and analyzing dimensional data on the surgical object;
(c) associating the collected and analyzed dimensional data with a reference model from a computer database associated with the tracking system;
(d) selecting the reference model; and
(e) generating information for performing the surgery based upon the selected reference model.

2. The method of claim 1, wherein the surgical object comprises a surgical implant or instrument.

3. The method of claim 1, wherein the surgical object is releasably coupled to a calibration device, the calibration device being detectable by the tracking system.

4. The method of claim 1, wherein the surgical object is releasably coupled to a tracker, the tracker being detectable by the tracking system.

5. The method of claim 1, wherein the surgical object is releasably coupled to a tracked instrument or object, the tracked instrument or object being detectable by the tracking system.

6. The method of claim 1, wherein step (c) comprises modifying the dimensions of the reference model to match the collected and analyzed dimensional data of the surgical object.

7. The method of claim 6, further comprising storing the modified dimensional data of the reference model in the computer database.

8. The method of claim 1, further comprising calibrating the surgical object by determining tip and axis information.

9. The method of claim 1, further comprising calibrating the surgical object by determining shape and geometry information.

10. The method of claim 1, wherein step (b) comprises exposing the surgical tool to a measurement field of the tracking system while the surgical tool contacts the surgical object.

11. The method of claim 1, further comprising generating a virtual image of the surgical object.

12. The method of claim 1, wherein the surgical tool comprises a surgical probe.

13. The method of claim 1, wherein step (d) is performed before step (b).

14. The method of claim 1, wherein a physician associates the collected and analyzed dimensional data with the reference model.

15. The method of claim 1, wherein software associates the collected and analyzed dimensional data with the reference model.

16. The method of claim 1, wherein a physician selects the reference model.

17. The method of claim 1, wherein software selects the reference model.

18. The method of claim 1, wherein the surgical information is provided by software.

19. The method of claim 1, wherein the surgical information comprises surgical planning information, anatomical resection information, sizing and rotational information, length and depth information or adjustment information for surgical devices and instruments.

20. A computer readable storage medium storing instructions that, when executed by a computer, causes the computer to perform a morphing process on a surgical object during a surgical navigation procedure, the morphing process comprising:
detecting a surgical tool with a tracking system when the surgical tool is exposed to a measurement field of the tracking system;
contacting the surgical tool at more than one point on the surgical object while tracking the surgical tool with the tracking system, thereby collecting and analyzing dimensional data on the surgical object;
associating the collected and analyzed dimensional data with a reference model from a computer database associated with the tracking system; and
generating information for performing the surgery based upon the selected reference model.

21. The computer readable storage medium of claim 20, wherein the morphing process further comprises:
generating a virtual image of the surgical object when the surgical tool is exposed to the measurement field of the tracking system;
modifying the dimensions of the reference model to match the collected and analyzed dimensional data of the surgical object; and
storing the modified dimensional data of the reference model in the computer database.

22. The computer readable storage medium of claim 20, wherein the morphing process further comprises selecting the reference model.

23. The computer readable storage medium of claim 20, wherein the morphing process further comprises calibrating the surgical object by determining tip and axis information.

24. The computer readable storage medium of claim 20, wherein the morphing process further comprises calibrating the surgical object by determining shape and geometry information.

25. The computer readable storage medium of claim 20, wherein the surgical object comprises a surgical implant or instrument.

26. The computer readable storage medium of claim 20, wherein the surgical tool comprises a surgical probe.

27. The computer readable storage medium of claim 20, wherein the surgical object is releasably coupled to a calibration device, the calibration device being detectable by the tracking system.

28. The computer readable storage medium of claim 20, wherein the surgical object is releasably coupled to a tracker, the tracker being detectable by the tracking system.

29. The computer readable storage medium of claim 20, wherein the surgical object is releasably coupled to a tracked instrument or object, the instrument or object being detectable by the tracking system.

30. A surgical navigation system, comprising:
a tracking system having a measurement field;
a surgical tool detectable by the tracking system when exposed to the measurement field;
means for collecting dimensional data from a surgical object while the surgical tool contacts the surgical object;
means for associating the surgical object with a reference model contained on a computer database;
means for selecting the reference model; and
means for generating information for performing the surgery when the reference model is selected.

31. The system of claim 30, further comprising:
means for generating a virtual image of the surgical object when the surgical tool is exposed to the measurement field of the tracking system;
means for modifying the dimensions of the reference model to match the collected dimensional data of the surgical object; and
means for storing the modified dimensional data of the reference model in the computer database.

32. The system of claim 30, further comprising calibrating the surgical object by determining tip and axis information.

33. The system of claim 30, further comprising calibrating the surgical object by determining shape and geometry information.

34. The system of claim 30, wherein the surgical object comprises a surgical implant or instrument.

35. The system of claim 30, wherein the surgical tool comprises a surgical probe.

36. The system of claim 30, wherein the surgical object is releasably coupled to a calibration device, the calibration device being detectable by the tracking system.

37. The system of claim 30, wherein the surgical object is releasably coupled to a tracker, the tracker being detectable by the tracking system.

38. The system of claim 30, wherein the surgical object is releasably coupled to a tracked instrument or object, the instrument or object being detectable by the tracking system.
